(19) United States
(12) Patent Application Publication
(10) Pub. No.: US 2013/0064819 A1
(43) Pub. Date: Mar. 14, 2013

(54) Biomarkers

(71) Applicants: Michael Binks, Stevenage (GB); Charles John Cox, Stevenage (GB); Marion Catherine Dickson, Stevenage (GB)

(72) Inventors: Michael Binks, Stevenage (GB); Charles John Cox, Stevenage (GB); Marion Catherine Dickson, Stevenage (GB)

(21) Appl. No.: 13/698,155

(22) PCT Filed: May 23, 2011
(86) PCT No.: PCT/EP2011/058324

§ 371 Date: Nov. 15, 2012

(30) Foreign Application Priority Data
May 25, 2010 (GB) 1008727.8

(51) Int. Cl. A61K 39/395; C40B 30/04; A61P 19/02; A61P 29/00; A61P 37/00; C12Q 1/68; C40B 40/06

(52) U.S. Cl. 424/134.1; 435/6.11; 506/9; 506/16; 424/133.1; 424/142.1

(57) ABSTRACT

The use of HLA-DQA1 as a biomarker for predicting or determining the therapeutic efficacy of anti-TNF therapy.

### FIELD OF THE INVENTION

The present invention relates to the use of biomarkers in the assessment and treatment of inflammation, particularly rheumatoid arthritis (RA) and in particular in the identification or prediction of those patients who will or will not respond to treatment with anti-TNF therapy, and methods and reagents for detecting such biomarkers.

### BACKGROUND OF THE INVENTION

The Human Leukocyte Antigen (HLA) system, also known as the human Major Histocompatibility Complex (MHC) is a cluster of linked genes located on chromosome 6, which is also known as the MHC region. The HLA system is classically divided into three regions: Class I, II, and III regions (Klein J. In: Gotze D, ed. The Major Histocompatibility System in Man and Animals, New York: Springer-Verlag, 1976: 339-378). Class I HLAs comprise a transmembrane protein (heavy chain) and a molecule of beta-2 microglobulin. The class I transmembrane proteins are encoded by the HLA-A, HLA-B and HLA-C loci. A function of class I HLA molecules is to present antigenic peptides (including viral protein antigens) to T cells. Three isoforms of class II MHC molecules, denoted HLA-DR, HLA-DQ, and HLA-DP are currently recognized. The MHC class II molecules have been implicated in the pathogenesis of a number of autoimmune diseases, due to their central roles in the presentation of antigenic peptides to helper T cells. The MHC class II molecules are heterodimers composed of an alpha chain and a beta chain; different alpha- and beta-chains are encoded by subsets of A genes and B genes, respectively. Various HLA-DR haplotypes have been recognized, and differ in the organization and number of DRB genes present on each DR haplotype; multiple DRB genes have been described. See Bodmer et al., Eur. J. Immunogenetics 24:105 (1997); Andersson, Frontiers in Bioscience 3:739 (1998). It is also clear that HLA-DQA and DQB genes exhibit polymorphisms in their coding and upstream regulatory regions (Del Pozzo et al 1992 Immunogenetics 25, 176-182). Such regions in DQB have been shown to confer different levels of expression on the genes under their control. The HLA-DQ locus has been associated with several autoimmune diseases including type I diabetes (She 1996 Immunology Today 17, 323, Baisch et al 1990 New England Journal of Medicine 95 6936) oligoarticular juvenile idiopathic arthritis (Ihle et al 2003 Clin Exp Rheumatologoly 21(2), 257-262) and dermatomyositis (Reed et al 1991 Human Immunology 32, 235)

The MHC region exhibits high polymorphism; more than 1200 alleles of HLA-B have been reported. See e.g., Schreuder et al., Human Immunology 60: 1157-1181 (1999); Bodmer et al., European Journal of Immunogenetics 26: 81-116 (1999). Despite the number of alleles at the HLA-A, HLA-B and HLA-C loci, the number of haplotypes observed in populations is smaller than mathematically expected. Certain alleles tend to occur together on the same haplotype, rather than randomly segregating. This association is called linkage disequilibrium (LD) and may be quantitated by methods that are known in the art (see, e.g., Devlin and Risch, Genomics 29:311 (1995); BS Weir, Genetic Data Analysis II, Sinauer Associates, Sunderland, Md. (1996)).

The products encoded by the polymorphic HLA loci may be typed by serological methods used in transplant and transfusion histocompatibility testing, and blood component therapy. Serological typing is based on reactions between characterized sera and HLA gene products. Known techniques for histocompatibility testing include, for example, microlymphocytotoxicity and flow cytometry. Standard microlymphocytotoxicity methodology for HLA antigen typing determines the HLA antigen profile of a subject's lymphocytes, using a panel of known HLA antisera. Certain HLA alleles are well characterized, and serologic methods of detecting them are known. See e.g., ASHI Laboratory Manual, Fourth Edition, American Society for Histocompatibility and Immunogenetics (2000); Hurley et al., Tissue Antigens 50:401 (1997).

Methods for analysis of HLA polymorphisms at the genetic level are also used. Non-serological HLA typing methods include the use of DNA restriction fragment length polymorphism (RFLP; see e.g., Erlich U.S. Pat. No. 4,582,788 (1986)), or labelled oligonucleotides, to identify specific HLA DNA sequences. Such methods may detect polymorphisms located in either the coding or noncoding sequence of the genome. See e.g., Bidwell et al, Immunology Today 9:18 (1988), Angelini et al., Proc. Natl. Acad. Sci. USA, 83:4489 (1986); Scharf et al., Science, 233:1076 (1986); Cox et al., Am. J. Hum. Gen., 43:954 (1988); Tiercy et al., Proc. Natl. Acad. Sci. USA 85:198 (1988); and Tiercy et al., Hum. Immunol. 24:1 (1989). The polymerase chain reaction (PCR) process (see, e.g. U.S. Pat. No. 4,683,202, 1987) allows amplification of genomic DNA and may be used for HLA typing procedures. See, e.g. Saiki et al., Nature 324:163 (1986); Bugawan et al., J. Immunol. 141:4024 (1988); Gyllensten et al., Proc. Natl. Acad. Sci. USA, 85:7652 (1988). See also e.g., Ennis et al., PNAS USA 87:2833 (1990); Petersdorf et al., Tissue Antigens 46: 77 (1995); Girdlestone et al., Nucleic Acids Research 18:6702 (1990); Marcos et al., Tissue Antigens 50:665 (1997); Steiner et al., Tissue Antigens 57:481 (2001); Madrigal et al., J. Immunology 149:3411 (1992).

Inflammation represents a group of vascular, cellular and neurological response to trauma. Inflammation can be characterised as the movement of inflammatory cells such as monocytes, neutrophils and granulocytes into the tissues. This is usually associated with reduced endothelial barrier function and oedema into the tissues. Inflammation can be classified as either acute or chronic. Acute inflammation is the initial response of the body to harmful stimuli and is achieved by the increased movement of plasma and leukocytes from the blood of injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

The aim of anti-inflammatory therapy is therefore to reduce this inflammation and allow for the physiological process of healing and tissue repair to progress.

Tumour necrosis factor (TNF) alpha sometimes known as tumour necrosis factor a (TNFα) is a cytokine which is included in the inflammatory response, which in turn causes many of the clinical sequelae associated with autoimmune disorders (Feldman, 2002, Nature Reviews Immunology, 2(5), 364-371). Therapeutic blockade of TNFα represents a significant advance in the treatment of patients with chronic inflammatory diseases, particularly rheumatoid arthritis (which is the most frequent form of chronic autoimmune arthritis, affecting 0.5-1% of the adult population), psoriatic arthritis, ankylosing spondylitis, juvenile chronic arthritis as well as other inflammatory conditions wherein anti-TNF therapies are prescribed.

Rheumatoid arthritis (RA) is characterised by joint pain, swelling and stiffness due to synovitis and irreversible joint destruction, ultimately leading to functional disability. RA is diagnosed based on fulfillment of the classification criteria set by the American College of Rheumatology (ACR). If at least 4 out of 7 criteria are fulfilled, the patient is diagnosed with RA. See Arnett et al 1988 Arthritis and Rheumatism 315-324). RA can occur in association with a chronic inflammatory disorder or can be considered new onset.

There are a number of other related “rheumatic” diseases with comparable inflammation of synovial tissue, including ankylosing spondylitis, psoriatic arthritis and some forms of osteoarthritis that can also be considered under the heading of chronic synovial inflammation.

The development of biological therapies that antagonise TNFα has transformed the treatment of rheumatoid arthritis, resulting in decreased morbidity and mortality and clinically meaningful improvement in quality of life (Feldman et al; 2008, Immunological Reviews, 223(1), 1-17). There are currently 4-5 marketed drugs (Enbrel (etanercept), Remicade (infliximab), Cimzia (certolizumab pegol), Simponi (golimumab) and Humira (adalimumab)). whose mechanism of action is through inhibiting the cytokine TNFα. In addition, many other biologicals including Rituxan (rituximab, anti CD20 antibody), Orencia (abatacept, anti CTLA-4 antibody) and Actemra and RoActemra (tocilizumab, anti-interleukin 6 receptor antibody) with different mechanisms are beginning to compete in this market area. To achieve maximum benefit of therapy on the signs and symptoms of disease with anti-TNF biologics can take 3-4 months. As a result, clinical treatment guidelines in the US and Europe recommend a minimum of 3-4 months treatment to determine therapeutic response.

As anti-TNF therapies are associated with potentially life-threatening or fatal adverse reaction [e.g. black box warnings for series infections which may lead to hospitalisation or death, lymphomas and other malignancies in children or adolescent patients] and as not all patients will respond to treatment, it would be desirable to improve the treatment of rheumatoid arthritis by providing a prognostic test to improve the initial assessment and subsequent treatment of RA, by specifying a patient population for anti-TNF treatment, in particular by estimating likely treatment response. Clinical studies clearly show that around 60% of patients prescribed with anti-TNF therapies do not achieve remission (Breedveld et al Arthritis and Rheumatism, 2006, 54(1), 26-37; Emery et al, Lancet, 2008, 372 (9636), 375-382; St Claire el al, Arthritis and Rheumatism, 2004, 50(11), 3432-3443) and 30% do not respond to the drug (change in DSA28≧1.2 or ACR20 achieved. If patients who are not likely to respond to anti-TNF therapy could be identified prior to treatment or during treatment, but earlier than a determination of non-response would typically be made, novel biologicals could then be tested in smaller, more efficient trials thus allowing such drugs to become first line therapies in these non-responder populations. Furthermore, it would be highly desirable to be able to select patients for anti-TNF therapy based on the likelihood they will respond to therapy and show improved disease activity, or alternatively will not likely respond and thus be moved to a different therapy. This is particularly desirable because the cost of such therapies is high and thus a significant burden on the healthcare system.

Ideally, a biomarker that predicts a response or non-response to anti-TNF therapy in RA or other chronic conditions could be measured in an easily obtainable biological sample (e.g. blood or other cells or tissue or saliva) from which DNA, RNA or protein can be analysed is desirable

Researchers have previously attempted to identify RNA markers predictive of responsiveness to anti-TN F therapies, (Lequerre et al, 2006, Arthritis research and Therapy, 8(4), art no, R105; Koczan et al, 2008, Arthritis Research and Therapy, 10(3), art no, R50), however these markers failed to replicate within the same or across different racial populations. Since TNFα plays such a fundamental role in the pathogenesis of RA many groups have studied the potential association between TNFα polymorphism and therapeutic response to anti TNF therapy. Several authors have found that −308 TNFα genotype (as detected by rs1600629) could be used to predict response to infliximab, etanercept or adalimumab (Mugnier et al Arthritis and Rheumatism 2003 48 1849-1852, Fonseca et al Annals of Rheumatic Disease 2005 64 793-794, Seitz et al Rheumatology 2007 46 93-96) however conflicting results from other groups have been reported (Criswell et al Arthritis and Rheumatism 2004 50 2750-2756, Marotte et al Annals of Rheumatic Disease 2006 65 342-347). Thus there remains a need for methods that can predict a patient's response to anti-TNF therapies, such as a test wherein the results of the test indicate a likelihood of response.

It is hence an aspect of the present invention to identify novel prognostic indicators to improve both an initial assessment and subsequent monitoring of RA patients. It is a further aspect of the present invention to specify a patient population for the treatment of RA, in particular by predicting treatment response. It is a further aspect of the present invention to improve success of treatment of RA. It is a further aspect of the present invention to improve success of treatment of RA with anti-TNF therapy. It is a further aspect of the present invention to identify patients that will respond to anti-TNF therapy.

### SUMMARY OF THE INVENTION

The present invention relates to the use of HLA-DQA1 as a biomarker for predicting or determining the therapeutic efficacy of anti-TNF therapies.

In particular, the invention provides that if an individual has certain genotypes from the Class II Major Histocompatibility Complex (MHC) gene DQA1, specifically the HLA-DQA1*05:01G genotype (encompassing the DQA1 alleles DQA1*04:01:01, 04:01:02, 04:02, 04:03 N, 04:04, 05:01:01, 05:01:02, 05:02, 05:03, 05:04, 05:05, 05:06, 05:07, 05:08, 05:09:05:10, 06:01:01, 06:01:02 and 06:02, which all have identical genomic sequence over the antigen binding domain, encoded by exon 2) and/or carry the A allele for RS9272535, an intronic SNP in the HLA-DQA1 gene, which acts as a proxy marker for individuals who carry an HLA- DQA1*04, 05 or 06 allele, then the presence of these genotypes is predictive of non-responsiveness to anti-TNF therapy.

In one aspect of the invention there is provided the use of HLA-DQA1 as a biomarker, in particular for determining the therapeutic efficacy of anti-TNF therapies.

In one aspect the present invention provides a method for identifying a patient as a likely responder/non-responder to anti-TNF therapy comprising determining the genotype of HLA-DQA1 of the patient.

In a further aspect, there is provided a method of treating inflammation or autoimmune disorders comprising determining the genotype of HLA-DQA1 of the patient and then administering an appropriate therapy.

In a further aspect the present invention there is provided a method of treating a patient who is likely to respond to anti-TNF therapy comprising determining the genotype of HLA-DQA1 of the patient followed by administering an anti-TNF therapeutic.

In a further aspect there is provide a method for treating a patient who is likely not to respond to ant-TNF therapy comprising determining the genotype of HLA-DQA1 of a patient followed by the administration of a non anti-TNF therapy, (e.g. anti-CD20 antibody, anti-CD52 antibody, anti-OSM antibody, anti-CD3 antibody, ofatumumab, etc).

In a further aspect there is provided an ex-vivo or in vitro method for determining the HLA-DQ1 genotype of a patient from a sample.

In a further aspect there is provided a diagnostic kit comprising means for determining the HLA-DQ1 genotype of a patient.

### DESCRIPTION OF THE FIGURES

FIG. 1:

Boxplot of baseline DAS28 ESR (Disease activity scores erythrocyte sedimentation rate) by anti-TNF therapy (Enbrel® (etanercept) [n=25], Humira™ (adalimumab) [n=37] and Remicade® (infliximab) [n=3])

FIG. 2:

A subset of 25 RA patients were classified into responders to anti-TNF treatment (change in DAS28-ESR) of ≧1.2 from baseline to Visit 5 (3 months following the initiation of anti TNF 15 therapy) or non responders to anti-TNF treatment (change in disease activity score (DAS28-ESR) of <1.2 baseline to Visit 5 (3 months following the initiation of anti TNF therapy). HLA-DQA1 mRNA was extracted from Pax tubes at baseline and the expression level measured on an Affymetrix U133A microarray. Expression level of each probe set was plotted and separated into responders and non-responders to any of the anti TNF therapies.

FIG. 3:

HLA-DQA1 mRNA expression as measured by Affymetrix probe 203290_at from whole blood of RA patients naive to anti-TNF treatment was plotted. Patients were subdivided into different disease activity based on DAS28-ESR score (Rem=remission DAS28<3.1. Mod=moderate DAS28 >3.1 and <5.1 and High DAS28 >5.1).

FIG. 4:

Frequency of DQA1*05:01G carriage in responders and non-responders in A) all treatment groups (n=63); B) etanercept treatment group (n=25) and C) combined adalimumab and 30 infliximab treatment groups (n=38)

### DETAILED DESCRIPTION OF THE INVENTION

The present invention relates to the discovery that the presence of certain genotypes or related phenotypes of HLA-DQA1 are predictive of whether a patient is likely to respond to anti-TNF therapy or is unlikely to respond to anti-TNF therapy.

Suitable anti-TNF agents include currently available agents (e.g. entacercept, inflixmab certalizumab, golimunab or adalimumab) but also encompass additional therapeutic agents that inhibit TNF.

The present invention provides methods for identifying patients that will likely respond to treatment with anti-TNF therapy. This allows for appropriate therapy to patients suffering from RA and a more efficient method to design clinical trials for both anti-TNF and non anti-TNF therapies.

In one aspect the patients suffer from inflammatory disorder and/or autoimmune disorders including rheumatoid arthritis, psoriatic arthritis, ankylosing spondylitis, juvenile chronic arthritis, or other disorders which may be treated with anti-TNF therapy.

In one aspect the patients have RA.

In one aspect, the patient is a human.

In one aspect there are provided methods of treatment of inflammation and/or autoimmune disease which comprises determining whether a patient will respond to treatment with anti-TNF therapy followed by the appropriate treatment. The appropriate treatment will depend on the outcome of the test for whether they are likely responders or not to anti-TNF therapy. In one aspect the disease to be treated is rheumatoid arthritis.

Examples of samples from a patient which may be used in the methods of the present invention include, for example, blood and blood products, synovial fluid, tissue, cell culture, hair, saliva, DNA, RNA or protein or derivatives thereof. In one aspect the sample is blood or blood products or derivatives thereof.

In one aspect the testing method of the present invention is performed before administration of therapy. As a result, only patients determined to be likely responsive to anti-TNF therapy are subject to treatment using an anti-TNF therapy so that efficacy can be increased and the incidence of any side effect can be decreased.

Moreover, a recommendation is indicated in product instructions such as an attached document for an anti-TNF therapy kinase inhibitor, such that the testing method of the present invention is performed before administration, so that it is administered to only patients likely to respond.

As is well known in genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, the “HLA allele” refers to one or more of the following alleles: HLA-DQA1*05:01G (encompassing alleles DQA1*04:01:01, 04:01:02, 04:02, 04:03 N, 04:04, 05:01:01, 05:01:02, 05:02, 05:03, 05:04, 05:05, 05:06, 05:07, 05:08, 05:09:05:10, 06:01:01, 06:01:02 and 06: and other markers in linkage disequilibrium with these alleles.

As used herein, “genotyping” a subject (or DNA or other biological sample) for a polymorphic allele of a gene(s) means detecting which allelic or polymorphic form(s) of the gene(s) or gene expression products (e.g., heterogeneous nuclear (hn) RNA, messenger RNA or protein) are present or absent in a subject (or a sample). Related RNA or protein expressed from such genes may also be used to detect polymorphic variation. As is well known in the art, an individual may be heterozygous or homozygous for a particular allele. More than two allelic forms may exist, thus there may be more than three possible genotypes. For purposes of the present invention, “genotyping” includes the determination of HLA alleles using suitable serologic, histologic or molecular techniques among others, as are known in the art. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g, sequencing) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with antiTNF therapies or individuals with RA.

An allele refers to one specific form of a genetic sequence (such as a gene) within a cell, a sample, an individual or within a population, the specific form differing from other forms of the same gene in the sequence of at least one, and frequently more than one, variant sites within the sequence of the gene. The sequences at these variant sites that differ between different alleles are termed “variants”, “polymorphisms”, or “mutations.” In general, polymorphism is used to refer to variants that have a frequency of at least 1% in a population, while the term mutation is generally used for variants that occur at a frequency of less than 1% in a population. In diploid organisms such as humans, at each autosomal specific chromosomal location or “locus” an individual possesses two alleles, a first inherited from one parent and a second inherited from the other parent, for example one from the mother and one from the father. An individual is “heterozygous” at a locus if it has two different alleles at the locus. An individual is “homozygous” at a locus if it has two identical alleles at that locus.

A polymorphism may comprise one or more of base changes, an insertion, a repeat, or a deletion. A polymorphic locus may be as small as one base pair. Polymorphic markers include restriction fragment length polymorphisms (RFLPs), variable number of tandem repeats, (VNTR's), hypervariable regions, minisatellites, dinucleotide repeats, trinucleotide repeats, tetranucleotide repeats, simple sequence repeats, and insertion elements such as Alu, among others. The first identified allelic form is arbitrarily designated as the reference form and other allelic forms are designated as alternative or variant alleles. The allelic form occurring most frequently in a selected population is sometimes referred to as the wildtype form. The most frequent allele may also be referred the major allele and the less frequent allele as the minor allele. Diploid organisms may be homozygous or heterozygous for allelic forms. A diallelic polymorphism has two forms. A triallelic polymorphism has three forms.

Single nucleotide polymorphisms (SNPs) are positions at which two alternative bases occur 35 at appreciable frequency (>1%) in the human population, and are the most common type of human genetic variation. Approximately 90% of all polymorphisms in the human genome are SNPs. SNPs are single base positions in DNA at which different alleles, or alternative nucleotides, exist in a population. An individual may be homozygous or heterozygous for an allele at each SNP position. A SNP can, in some instances, be referred to as a “cSNP” to denote that the nucleotide sequence containing the SNP is an amino acid coding sequence. As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001).

Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. cSNPs or coding SNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in Lariat regions, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence which leads to an increased risk of disease, phenotype or therapeutic response. In this situation, the presence of a SNP correlates with the presence of, or predisposition to, or an increased risk in developing the disease, phenotype or therapeutic response. These SNPs, although not causative, are nonetheless also useful for diagnostics, therapeutic response markers, disease predisposition screening, and other uses.

An association study of a SNP and a specific disorder or a predisposition to therapeutic response or risk of an adverse event involves determining the presence or frequency of the SNP allele in biological samples from individuals with the disorder or predisposition to therapeutic response or risk of an adverse event of interest and comparing the information to that of controls (i.e. individuals who do not have the disorder, do not respond to therapy or who do not experience the adverse event).

A SNP may be screened in diseased tissue samples or any biological sample obtained from an individual that contains genetic information, and compared to control samples, and selected for its increased (or decreased) occurrence in a specific pathological condition or therapeutic response. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype.

Clinical trials have shown that patient response to treatment with pharmaceuticals is often heterogeneous. There is a continuing need to improve pharmaceutical agent design and therapy. In that regard, SNPs can be used to identify patients most suited to therapy with particular pharmaceutical agents (this is often termed “pharmacogenomics”). Similarly, SNPs can be used to exclude patients from certain treatment due to the patient's increased likelihood of developing toxic side effects or their likelihood of not responding to the treatment. Pharmacogenomics can also be used in pharmaceutical research to assist the drug development and selection process. (Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 3).

Several techniques for the detection of mutations have evolved based on the principal of hybridization analysis. For example, in the primer extension assay, the DNA region spanning the nucleotide of interest is amplified by PCR, or any other suitable amplification technique. After amplification, a primer is hybridized to a target nucleic acid sequence, wherein the last nucleotide of the 3′ end of the primer anneals immediately 5′ to the nucleotide position on the target sequence that is to be analyzed. The annealed primer is extended by a single, labelled nucleotide triphosphate. The incorporated nucleotide is then detected.

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The Protein Truncation Test (PTT) is also commonly used to detect genetic polymorphisms (Roest et al., Human Molecular Genetics 2:1719-1721, (1993); Van Der Luit et al., Genomics 20:1-4 (1994); Hogervorst et al., Nature Genetics 10: 208-212 (1995)). Typically, in the PTT, the gene of interest is PCR amplified, subjected to in vitro transcription/translation, purified, and analyzed by polyacrylamide gel electrophoresis.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

$$r = \frac{{\hat{Δ}}_{AB}}{\sqrt{( {\tilde{π}}_{A} + {\hat{D}}_{A} ) ( {π}_{B} + {D}_{B} )}}$$

$$where$$

$${\tilde{π}}_{A} = {\tilde{p}}_{A} ( 1 - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2}$$

$${\hat{Δ}}_{AB} = \frac{1}{n} {n}_{AB} - 2 {\tilde{p}}_{A} {\tilde{p}}_{B}$$

nr² has an approximate chi square distribution with 1 degree freedom for biallelic markers. Loci exhibiting an r such that nr² is greater than 3.84, corresponding to a significant chi-squared statistic at the 0.05 level, are considered to be in linkage disequilibrium (BS Weir 1996 Genetic Data Analysis II Sinauer Associates, Sunderland, Md.).

Alternatively, a normalized measure of linkage disequilibrium can be defined as:

$${D}_{AB}^{/} = \{ \begin{matrix}\frac{{D}_{AB}}{min ( {p}_{A} {p}_{B} , {p}_{a} {p}_{b} )} , & {D}_{AB} < 0 \\ \frac{{D}_{AB}}{min ( {p}_{A} {p}_{b} , {p}_{a} {p}_{B} )} , & {D}_{AB} > 0\end{matrix}$$

The value of the D′ has a range of −1.0 to 1.0. When statistically significant absolute D′ value for two markers is not less than 0.3 they are considered to be in linkage disequilibrium.

As used herein the word “haplotype” refers to a set of closely linked HLA alleles present on one chromosome which tend to be inherited together.

The HLA-A*01:01, -C*07:01, -B*08:01, -DRB1*03:01, DQA1*05:01, DQB1*02:01 combination of HLA genotypes is referred to as the HLA A1-B8-DR3-DQ2 haplotype. An HLA genotype can be identified by detecting the presence of an HLA allele, or detecting a genetic marker known to be in linkage disequilibrium with an HLA allele. A genotype refers to variation at a defined position in a single gene, e.g., 1,1 1,2 2,2. DQA1, DQB1 and DRB1 are distinct genes and code for different proteins.

As used herein, determination of a ‘multilocus’ genotype (also known as a haplotype) refers to the detection within an individual of the alleles present at more than one locus. A subject may be genetically screened to determine the presence or absence of both an HLA allele (e.g., an HLA-DQA1*05:01G) and another allele, e.g, a different HLA allele or a non-HLA allele.

As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

Alleles, polymorphisms or genetic markers that are ‘associated’ with non-response to anti-TNF therapy, in that they have been found to be over-represented in frequency in populations of treated subjects that do not experience a significant decrease (<1.2) in DAS 28 score, as compared to individuals with a significant decrease in DAS 28 score.

Accordingly, the present invention relates to the discovery that the presence of HLA-DQA1*05:01G genotype and/or the presence of the A allele for RS9272535 provides a measure that predicts a patient's likelihood of response to anti-TNF therapy. Those individuals that have a DQA1*05:01G genotype and/or carry the A allele for RS9272535 are less likely to respond to anti-TNF therapy than patients who do not carry either of these alleles. The A allele for RS9272535 acts as a proxy marker for a subset of HLA DQA1 alleles, specifically, DQA1*04:01:01, 04:01:02, 04:02, 04:03 N, 04:04, 05:01:01, 05:01:02, 05:02, 05:03, 05:04, 05:05, 05:06, 05:07, 05:08, 05:09:05:10, 06:01:01, 06:01:02 and 06:02, in that an individual found to carry at least one copy of the A allele for RS9272535 will also carry one of the aforementioned DQA1 alleles.

Thus, genotyping the HLA-DQ1 gene is intended to encompass both genotyping for the HLADQA*05:01G genotype and/or RS9272535.

In one aspect the genotype of the HLA-DQA1 gene and/or RS9272535 is measured by determining the DNA polynucleotide sequence, or by detecting the corresponding sequence in RNA transcripts from the polymorphic gene, or where the nucleic acid polymorphism results in a change in an encoded protein by detecting such amino acid sequence changes in encoded proteins; using any suitable technique as is known in the art. Polynucleotides utilized for typing are typically genomic DNA, or a polynucleotide fragment derived from a genomic polynucleotide sequence, such as in a library made using genomic material from the individual (e.g. a cDNA library). The polymorphism may be detected in a method that comprises contacting a polynucleotide or protein sample from an individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In the case where the presence of the polymorphism is being determined in a polynucleotide it may be detected in the double stranded form, but is typically detected in the single stranded form.

The binding agent may be a polynucleotide (single or double stranded) typically with a length of at least 10 nucleotides, for example at least 15, 20, 30, or more nucleotides. A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region. The binding agent may be a molecule that is structurally similar to polynucleotides that comprises units (such as purine or pyrimidine analogs, peptide nucleic acids, or RNA derivatives such as locked nucleic acids (LNA)) able to participate in Watson-Crick base pairing. The agent may be a protein, typically with a length of at least 10 amino acids, such as at least 20, 30, 50, or 100 or more amino acids. The agent may be an antibody (including a fragment of such an antibody that is capable of binding the polymorphism).

In one embodiment of the present methods a binding agent is used as a probe. The probe may be labelled or may be capable of being labelled indirectly. The detection of the label may be used to detect the presence of the probe on (bound to) the polynucleotide or protein of the individual. The binding of the probe to the polynucleotide or protein may be used to immobilize either the probe or the polynucleotide or protein (and thus to separate it from one composition or solution).

In another embodiment of the invention the polynucleotide or protein of the individual is immobilized on a solid support and then contacted with the probe. The presence of the probe immobilized to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The present methods may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In one embodiment the probe is used in a heteroduplex analysis based system to detect polymorphisms. In such a system when the probe is bound to a polynucleotide sequence containing the polymorphism, it forms a heteroduplex at the site where the polymorphism occurs (i.e. it does not form a double strand structure). Such a heteroduplex structure can be detected by the use of an enzyme that is single or double strand specific. Typically the probe is an RNA probe and the enzyme used is RNAse H that cleaves the heteroduplex region, thus allowing the polymorphism to be detected by means of the detection of the cleavage products.

The method may be based on fluorescent chemical cleavage mismatch analysis which is described for example in PCR Methods and Applications 3:268-71 (1994) and Proc. Natl. Acad. Sci. 85:4397-4401 (1998).

In one embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual, and the presence of the polymorphism is determined by the detection of the PCR product. Preferably the region of the primer which is complementary to the polymorphism is at or near the 3′ end the primer. In one embodiment of this system the polynucleotide agent will bind to the wild-type sequence but will not act as a primer for a PCR reaction.

The method may be a Restriction Fragment Length Polymorphism (RFLP) based system. This can be used if the presence of the polymorphism in the polynucleotide creates or destroys a restriction site that is recognized by a restriction enzyme. Thus treatment of a polynucleotide that has such a polymorphism will lead to different products being produced compared to the corresponding wild-type sequence. Thus the detection of the presence of particular restriction digest products can be used to determine the presence of the polymorphism.

The presence of the polymorphism may be determined based on the change that the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DGGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the TAQMAN™ PCR detection system. In another method of detecting the polymorphism a polynucleotide comprising the polymorphic region is sequenced across the region which contains the polymorphism to determine the presence of the polymorphism.

Various other detection techniques suitable for use in the present methods will be apparent to those conversant with methods of detecting, identifying, and/or distinguishing polymorphisms. Such detection techniques include but are not limited to direct sequencing, use of “molecular beacons” (oligonucleotide probes that fluoresce upon hybridization, useful in real-time fluorescence PCR; see e.g., Marras et al., Genet Anal 14:151 (1999)); electrochemical detection (reduction or oxidation of DNA bases or sugars; see U.S. Pat. No. 5,871,918 to Thorp et al.); rolling circle amplification (see, e.g., Gusev et al., Am J Pathol 159:63 (2001)); Third Wave Technologies (Madison Wis.) INVADER® non-PCR based detection method (see, e.g., Lieder, Advance for Laboratory Managers, 70 (2000))

Accordingly, any suitable detection technique as is known in the art may be utilized in the present methods.

As used herein, “determining” a subject's genotype does not require that a genotyping technique be carried out where a subject has previously been genotyped and the results of the previous genetic test are available; determining a subject's genotype accordingly includes referring to previously completed genetic analyses.

As used herein, a subject that is “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

An allele refers to one specific form of a genetic sequence (such as a gene) within a cell, a sample, an individual or within a population, the specific form differing from other forms of the same gene in the sequence of at least one, and frequently more than one, variant sites within the sequence of the gene. The sequences at these variant sites that differ between different alleles are termed “variants”, “polymorphisms”, or “mutations.” In general, polymorphism is used to refer to variants that have a frequency of at least 1% in a population, while the term mutation is generally used for variants that occur at a frequency of less than 1% in a population. In diploid organisms such as humans, at each autosomal specific chromosomal location or “locus” an individual possesses two alleles, a first inherited from one parent and a second inherited from the other parent, for example one from the mother and one from the father. An individual is “heterozygous” at a locus if it has two different alleles at the locus. An individual is “homozygous” at a locus if it has two identical alleles at that locus.

A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. The most frequent allele may also be referred the major allele and the less frequent allele as the minor allele. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (>1%) in the human population, and are the most common type of human genetic variation. Approximately 90% of all polymorphisms in the human genome are SNPs. SNPs are single base positions in DNA at which different alleles, or alternative nucleotides, exist in a population. An individual may be homozygous or heterozygous for an allele at each SNP position. A SNP can, in some instances, be referred to as a “cSNP” to denote that the nucleotide sequence containing the SNP is an amino acid coding sequence. As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001).

Several techniques for the detection of mutations have evolved based on the principal of hybridization analysis. For example, in the primer extension assay, the DNA region spanning the nucleotide of interest is amplified by PCR, or any other suitable amplification technique. After amplification, a primer is hybridized to a target nucleic acid sequence, wherein the last nucleotide of the 3′ end of the primer anneals immediately 5′ to the nucleotide position on the target sequence that is to be analyzed. The annealed primer is extended by a single, labelled nucleotide triphosphate. The incorporated nucleotide is then detected.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The Protein Truncation Test (PTT) is also commonly used to detect genetic polymorphisms (Roest et al., Human Molecular Genetics 2:1719-1721, (1993); Van Der Luit et al., Genomics 20:1-4 (1994); Hogervorst et al., Nature Genetics 10: 208-212 (1995)). Typically, in the PTT, the gene of interest is PCR amplified, subjected to in vitro transcription/translation, purified, and analyzed by polyacrylamide gel electrophoresis.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

The present invention also provides for a predictive (patient care) test or test kit. Such a test will aid in the therapeutic use of pharmaceutical compounds, including anti-TNF therapies, based on pre-determined associations between genotype and phenotypic response to the therapeutic compound. Such a test may take different formats, including:
(a) a test which analyzes DNA or RNA for the presence of pre-determined alleles and/or polymorphisms. An appropriate test kit may include one or more of the following reagents or instruments: an enzyme able to act on a polynucleotide (typically a polymerase or restriction enzyme), suitable buffers for enzyme reagents, PCR primers which bind to regions flanking the polymorphism, a positive or negative control (or both), and a gel electrophoresis apparatus. The product may utilise one of the chip technologies as described by the state of the art. The test kit would include printed or machine readable instructions setting forth the correlation between the presence of a specific genotype and the likelihood that a subject treated with a specific pharmaceutical compound will experience a hypersensitivity reaction;
(b) a test which analyses materials derived from the subject's body, such as proteins or metabolites, that indicate the presence of a pre-determined polymorphism or allele. An appropriate test kit may comprise a molecule, aptamer, peptide or antibody (including an antibody fragment) that specifically binds to a predetermined polymorphic region (or a specific region flanking the polymorphism). The kit may additionally comprise one or more additional reagents or instruments (as are known in the art). The test kit would also include printed or machine-readable instructions setting forth the correlation between the presence of a specific polymorphism or genotype and the likelihood that a subject treated with a specific synthetic nucleoside analog will experience a hypersensitivity reaction.

Primers, probes, antibodies and other detection reagents specific for detecting HLA-DQA1*05:01G genotype or mRNA expression of exon 5 of the HLA-DQA1 gene and the genotype for RS9272535, as well as a kits or packs comprising at least one of these reagents, are also embodiments of the invention.

Suitable biological specimens for testing are those which comprise cells and DNA and include, but are not limited to blood or blood components, dried blood spots, urine, buccal swabs and saliva. Suitable samples for HLA serologic testing are well known in the art.

The testing method of the present invention is conveniently performed before administration of therapy. As a result, only patients determined to be responsive to anti-TNF therapy are subject to treatment using an anti-TNF therapy so that efficacy can be increased and the overall risk of adverse events in patients who are unlikely to respond can be reduced.

Moreover, a recommendation is indicated in product instructions such as an attached document for an anti-TNF therapy, such as that the testing method of the present invention is performed before administration, so that it is administered to only patients likely to respond. An example of the test kit for testing the likelihood of response to anti-TNF therapy, wherein the sensitivity is tested with the presence of the HLA-DQA1*05:01G genotype contained in samples of patients, is a kit.

An example of the test kit for testing the likelihood of response to anti-TNF therapy, wherein the sensitivity is tested with the presence of the A allele for SNP rs9272535.

An example of the test kit for testing the likelihood of response to anti-TNF therapy, wherein the sensitivity is tested with the use of the HLA-DQA1 gene contained in samples of patients, is a kit containing several primer pair sets specific to a reference sequence of exon 5 of the HLA-DQA1*05:01G alleles and a reagent thought to be necessary for gene amplification.

### EXAMPLES

### Description of Present Study.

GSK sponsored study RES11121 was designed to aid in the identification of novel markers in response to anti-TNF treatment of Rheumatoid Arthritis (RA), investigating genetic and genomic biomarkers.

Sixty eight RA patients naïve to anti-TNF therapy were recruited at a single study site (Royal Hallamshire Hospital, Sheffield, S10 2JF, United Kingdom). Patients were eligible for inclusion into the study if they provided written informed consent, were ≧18 years of age, met 1987 American College of Rheumatology criteria for the diagnosis of RA, they fulfilled NICE criteria ie they had to have previously failed to disease modifying anti-inflammatory drugs (DMARDs) and had anti-TNF therapy prescribed by their physician. An RA patient was not eligible for inclusion in this study if they were known to have HIV, Hepatitis B or C infection, or had received a blood transfusion within 4 weeks prior to enrolment. A baseline blood and urine sample was taken at first visit. Over a 14-16 week treatment period with anti-TNF therapy [etanercept(N=25), adalimumab (N=40) or infliximab (N=3)] blood and urine samples were collected at each of four follow up visits. Clinical assessment of RA was included in all visits.

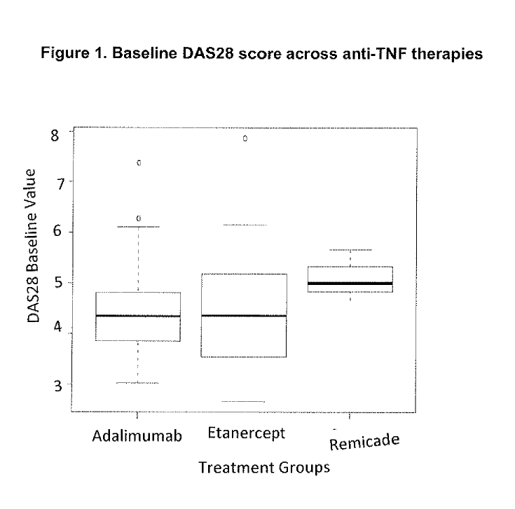

Baseline DAS28 ESR score was compared between the three treatment groups (see FIG. 1), with no significant differences seen between treatments. For the purposes of this pharmacogenetic study, results from patients treated with adalimumab or infliximab were combined as the mechanism of action of these monoclonal antibodies (mAb) are similar.

All subjects were British Caucasians. Sixty five patients consented to pharmacogenetics investigation, and had responder/non-responder information available (see Table 1). Forty-three patients responded to anti-TNF therapy (as defined by change from baseline (pre-treatment) in DAS28ESR score 1.2 following 14-16 weeks of anti-TNF therapy and twenty two patients did not show a change in DAS28ESR>1.2.

TABLE 1
anti-TNF Treatment Response Results

| | | VISIT 5-VISIT 1 | | |
|---|---|---|---|---|
| | | Adalimumab | Etanercept | Infliximab |
| | BASELINE | (Humira) | (Enbrel) | (Remicade) |
| Number of | 68 | 40 | 25 | 3 |
| Patients | | | | |
| Number of | 65 | 37 (24:13) | 25 (17:8) | 3 (2:1) |
| patients with | | | | |
| DNA+ treatment | | | | |
| outcome | | | | |
| information | | | | |
| (Responder:non- | | | | |
| responder) | | | | |

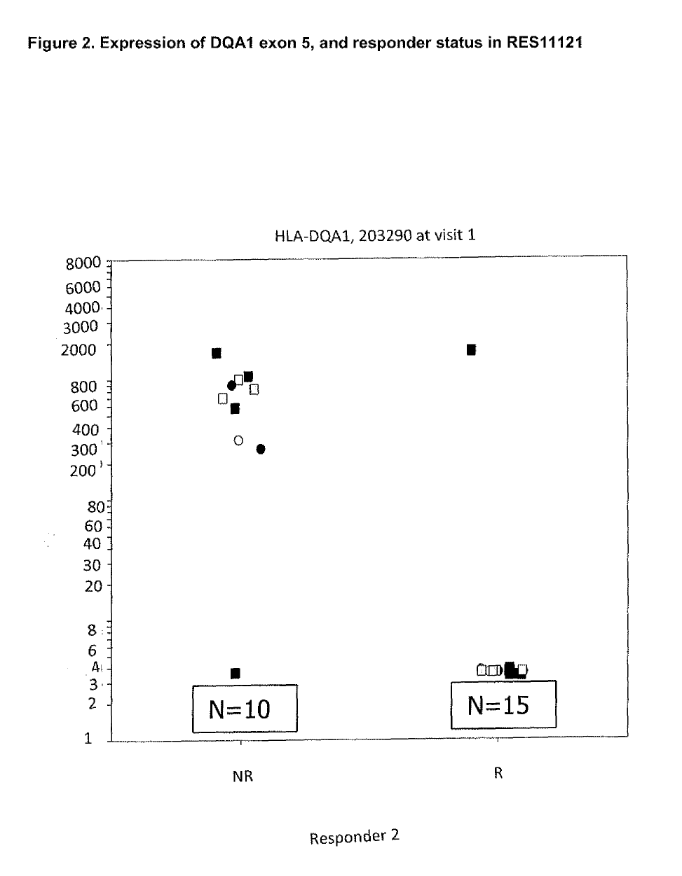

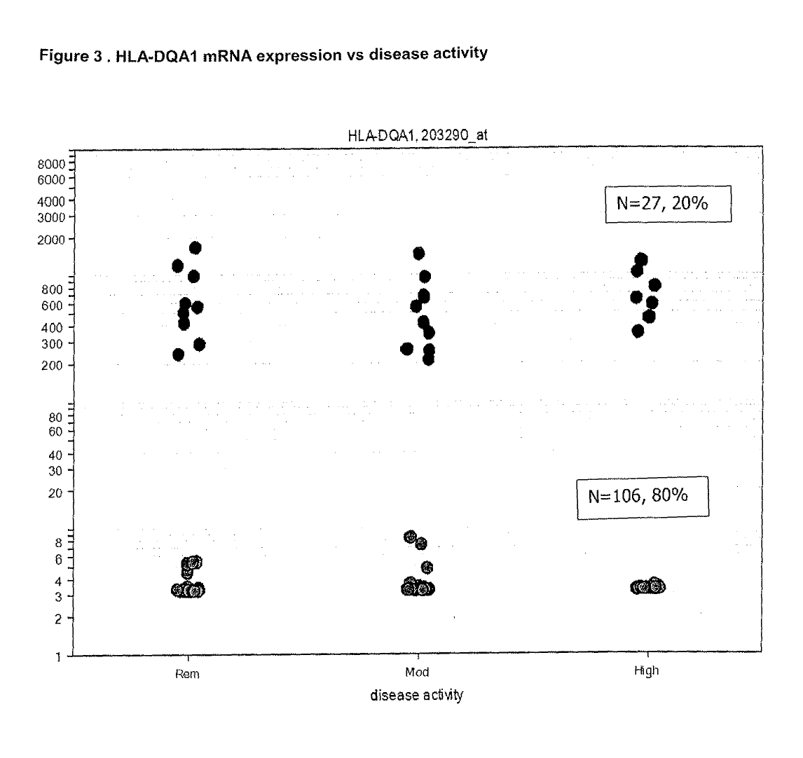

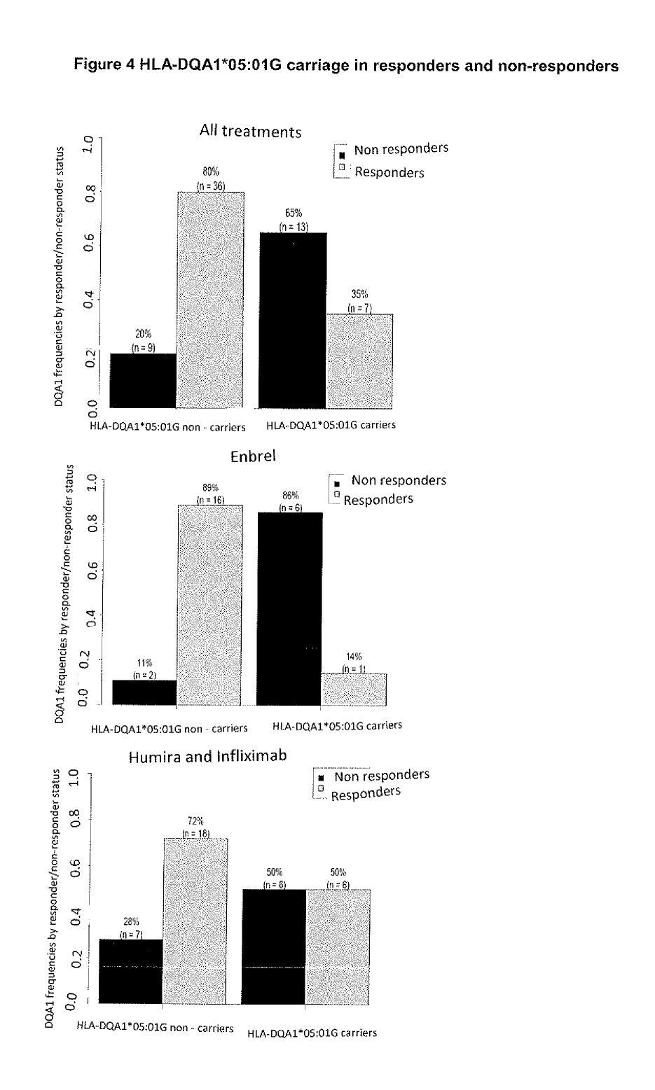

From within the same population transcriptomic analysis was performed on a subset of 25 RA patients, 15 of whom responded to the therapy (ΔDAS28>1.2 at 14-16 weeks vs baseline) and 10 did not. mRNA was extracted from PAX tubes collected prior to any anti-TNF therapy. mRNA was reverse transcribed and amplified using NuGen Ovation® RNA Amplification System V2 (NuGEN Technologies, Inc, San Carlos, CA, USA), and resulting patients cDNA fragmented, biotin-labelled and hybridised onto U133plus2 chips (Affymetrix, Santa Clara, Calif., USA). Data was analysed in Array Studio (v1.1) using a mixed model. ANOVA and t-tests were performed on all responders vs all non responders to the anti-TNF therapy. This investigation identified a single genomic marker (203290_at), homologous to the target sequence of HLA-DQA1 exon 5, which predicted the response to anti-TNF therapy (etanercept or adalimumab) with a sensitivity of 89% and a specificity of 93%. IDetection of expression for this marker in the RA patient indicates the patient is unlikely to respond to anti-TNF therapy. See FIG. 2.

This binary expression of the marker was also detected in a subset of another RA population (as described in Rioja et al Arthritis and Rheumatism 2008 56, 8, 2257-2267 see FIG. 3) studied and was not associated with disease activity status as defined as remission (DAS28<3.1), moderate (DAS28>3.1 and <5.1), or high (DAS28>5.1). For approximately 20% of the patients, expression of the mRNA from exon 5 of DQA1 was detected, suggesting that this set of patients may be less likely to respond to anti-TNF therapy.

This second RA population was also screened with a high-density set of 2,360 SNPs (Vignal et al Arthritis and Rheumatism 2009 60, 53-62) spanning the MHC region by Illumina (San Diego, Calif., USA). This screen constituted two panels, namely, the MHC Mapping Panel and the MHC Exon-Centric Panel. The former comprises evenly spaced SNP markers with, on average, 3.8 kb between each SNP; the latter focuses on SNP markers near or within exons. Review of data from this panel of markers identified a SNP (RS9272535) within the first intron of HLA-DQA1 that was 100% predictive of the HLA-DQA1 exon 5 expression profile. An individual with either an AA or AG genotype for RS9272535 was always seen to have an expression signal for expression marker 203290_at. If the subject carried a G/G genotype then the probe set representing exon 5 was not detected. This data was highly suggestive that the expression signal identified was driven by HLA-DQA1 genotype. Review of the probe sequence associated with 203290_at and exon 5 sequence from different DQA1 alleles highlighted that these probes were unlikely to detected expression from individuals that did not carry at least one copy of DQA1*04, *05 or *06 due to significant sequence differences between probe sequence and exon 5 of other alleles. Therefore it was hypothesised that the expression signal identified as being associated with non-response was due to the presence of either DQA1*04, DQA1*05 or DQA1*06 genotypes, and that high resolution HLA genotyping of DQA1 should be performed to further investigate this signal.

Germline DNA was extracted from peripheral blood (QiAmp DNA Blood Kit, Qiagen, Valencia, Calif.). Four digit classical HLA genotyping was performed for HLA-DQA1 and HLA-DRB1 at Histogenetics (Ossining, N.Y., USA), and interrogation of rs9272535 (Intron 1 HLA-DQA1) and RS1800629 (TNFα-308) using TaqMan® SNP genotyping assays (Applied Biosystems, Foster City, Calif., USA) performed at GlaxoSmithKline (Research Triangle Park, NC, USA).

Shared Epitope (SE) status (SE+ or SE−) was determined using HLA-DRB1 alleles. The SE+ alleles correspond to amino acid sequences QKRAA, QRRAA, or RRRAA at positions 70-74 of the third hypervariable region of the HLA-DRB1 molecule. From the four digit genotypes returned, HLA-DRB1 alleles 01:01:01, 01:02:01, 04:01:01, 04:04, 04:05:01, 35 04:08, and 10:01:01 were classed as SE+. HLA-DRB1 alleles 03:01:01, 04:02, 07:01, 11:01, 11:04:01, 12:01G, 13:01:01, 13:02:01, 13:03:01, 14:01G, 15:01:01 and 16:01:01 were classed as SE−.

Following high resolution HLA typing for HLA-DQA1, individual genotypes were identified that were part of HLA-DQA1*04/*05 or *6 serotypes. In the RES11121 population, only the HLA-DQA1*05:01G genotype was identified that belonged to these serotypes, as none of the individuals typed were positive for any HLA-DQA1*04 or *06 alleles, or DQA1*05 alleles other than DQA1*05:01G. As such, individuals were classified as HLA-DQA1*05:01G positive or negative prior to analysis.

### Analysis Plan:

In this study, two endpoints were considered: the change from baseline in DAS28 score and the responder status to anti-TNFα therapies, where responders were subjects with a change in DAS28 score ≧1.2 between baseline and 14-16 weeks of treatment. The statistical analyses were performed on data from patients treated with etanercept and the combined data from patients treated with either adalimumab and infliximab and the combined etanercept adalimumab and infliximab treatment groups. HLA-DQA1*05:01G was re-coded as a bi-allelic marker according to the presence/absence of the *05:01G alleles. Subjects carrying *05:01G/*05:01G or *05:01G/X, where X denotes all alleles except *05:01G, were coded as 1 and those who do not carry 05:01G, i.e. X/X, were coded as 0. The presence of allele A of the SNP marker rs9272535 was in complete LD with the presence of HLA-DQA1*05:01G (r²=1). Association analyses were hence performed considering the effect of HLA-DQA1*05:01G.

### Results:

A significant association (P_{non-resp/resp}=8.0×10⁻⁴, OR=7.4 [2.3, 24.0], PPV=0.65, NPV=0.80, SENS=0.59, SPEC=0.84) was seen between the presence of the DQA1*05:01G genotype and non-response to all anti-TNF therapies in this study (see table 2). Assessment of different treatment groups revealed a significant association between the presence of the DQA1*05:01G genotype and non-response to etanercept (P_{non-resp/resp}=0.003, OR=48.0 [3.6, 631.8], PPV=0.86, NPV=0.89, SENS=0.75, SPEC=0.94) which was not seen with the patients receiving adalimumab and infliximab (P_{non-resp/resp}=0.089, OR=3.3 [0.8, 13.4]) As can be seen from FIG. 4A, 65% of DQA1*05:01G carriers do not respond to all anti-TNF treatments, compared to 86% of patients given Enbrel (FIG. 4B) and only 50% patients given Adalimumab or Remicade (FIG. 4C).

TABLE 2
The effect of DQA1*05:01G and the risk of
being a non-responder to anti-TNF therapy

Unadjusted model

| | P | OR 95% CI |
|---|---|---|
| All treatments | 8.0 × 10⁻⁴ | 7.4 [2.3, 24.0] |
| | | PPV = 0.65 [0.41, 0.84] |
| | | NPV = 0.80 [0.65, 0.90] |
| | | SENS = 0.59 [0.36, 0.79] |
| | | SPEC = 0.84 [0.69, 0.93] |
| Etanercept | 0.003 | 48 [3.6, 631.8] |
| | | PPV = 0.86 [0.42, 0.99] |
| | | NPV = 0.89 [0.65, 0.98] |
| | | SENS = 0.75 [0.35, 0.97] |
| | | SPEC = 0.94 [0.71, 0.99] |
| adalimumab & | 0.089 | 3.3 [0.8, 13.4] |
| Infliximab | | PPV = 0.54 [0.25, 0.81] |
| | | NPV = 0.74 [0.54, 0.89] |
| | | SENS = 0.50 [0.23, 0.77] |
| | | SPEC = 0.77 [0.56, 0.911 |

PPV = positive predictive value, NPV = negative predictive value, SENS = Sensitivity, SPEC = specificity, OR = odds ratio.

To assess the impact of confounding factors, the model was adjusted to account for baseline DAS28 score and rs1600629 genotype, where the A allele has been shown to be associated with non-response (O'Rielly et al, The Pharmacogenetics Journal (2009) 9, 161-167). In RES11121 rs1600629 the −308 TNFα marker shown previously to be associated with non-response to anti TNF therapy, was again significantly associated with non-response to all treatments, and etanercept and adalimumab & infliximab treatments separately (see table 3). Performance characteristics of this marker between these groups did not differ significantly.

TABLE 3
The effect of the A allele of rs1600629 and the
risk of being a non-responder to anti-TNF therapy

Unadjusted model

| | P | OR 95% CI |
|---|---|---|
| All treatments | 6.0 × 10⁻⁴ | 7.6 [2.4, 24.2] |
| | | PPV = 0.64 [0.41; 0.83] |
| | | NPV = 0.81 [0.67; 0.92] |
| | | SENS = 0.64 [0.41; 0.83] |
| | | SPEC = 0.81 [0.67; 0.92] |
| Etanercept | 0.034 | 7.8 [1.2, 51.9] |
| | | PPV = 0.62 [0.24; 0.91] |
| | | NPV = 0.82 [0.56; 0.96] |
| | | SENS = 0.62 [0.24; 0.91] |
| | | SPEC = 0.82 [0.56; 0.96] |
| Adalimumab & | 0.007 | 7.6 [1.7, 32.7] |
| Infliximab | | PPV = 0.64 [0.35; 0.87] |
| | | NPV = 0.81 [0.61; 0.93] |
| | | SENS = 0.64 [0.35; 0.87] |
| | | SPEC = 0.81 [0.61; 0.93] |

PPV = positive predictive value, NPV = negative predictive value, SENS = Sensitivity, SPEC = specificity, OR = odds ratio.

There was no significant association at a type-I error α=0.05 between the number of allele copies of the shared epitope and the risk of being a non-responder in Etanercept, the combined Adalimumab and Remicade group or in the combined treatment groups.

Logistic regression was used to investigate genetic association between the presence of HLA-DQA1*05:01G and the risk of being a non-responders, including baseline DAS28 score, and baseline DAS28 score and rs1600629 as confounding factors (see table 4).

TABLE 4
Association results of the presence of DQA1*05:01G
and the risk of being a non responder

| | | | | | Model adjusted on baseline | |
|---|---|---|---|---|---|---|
| | | | Model adjusted on | | DAS28 score | |
| | Unadjusted model | | baseline DAS28 score | | and TNF rs1800629 | |
| | P | OR 95% Cl | P | OR 95% Cl | P | OR 95% Cl |
| All | 8.0 × 10⁻⁴ | 7.4 [2.3, 24.0] | 7.0 × 10⁻⁴ | 12.5 [2.9, 54.5] | 0.014 | 7.2 [1.5; 31.2] |
| treatments | | | | | | |
| Etanercept | 0.003 | 48 [3.6, 631.8] | 0.006 | 55.5 [3.1; >999.9] | 0.019 | 38.5 [1.8; 809.4] |
| Adalimumab & | 0.089 | 3.3 [0.8, 13.4] | 0.039 | 6.6 [1.1, 39.3] | 0.224 | 3.4 [0.5, 24.5] |
| Infliximab | | | | | | |

HLA-DQA1*05:01G maintained a significant association in the all treatment group and in the subgroup of patients who received etanercept.

Additionally, logistic regression was used to investigate potential genetic associations between the presence of the A allele of rs1600629 and the risk of being a non-responder, including baseline DAS28 score, and baseline DAS28 score and HLA-DQA1*05:01G genotype as confounding factors (see table 5).

TABLE 5
Association results of the presence of allele A at rs1800629 and the risk of
being a non-responder to antiTNF therapy

| | | | | | Model adjusted on baseline | |
|---|---|---|---|---|---|---|
| | | | Model adjusted on baseline | | DAS28 score and the presence | |
| | Unadjusted model | | DAS28 score | | of DQA1*05:01G | |
| | P | OR [95% Cl] | P | OR [95% Cl] | P | OR [95% Cl] |
| All | 6 × 10⁻⁴ | 7.6 [2.4; 24.4] | 0.001 | 8.4 [2.3; 30.6] | 0.044 | 4.3 [1.0; 18.2] |
| treatments | | | | | | |
| Etanercept | 0.034 | 7.8 [1.2; 51.9] | 0.042 | 8.8 [1.1; 71.1] | 0.489 | 2.8 [0.1; 51.3] |
| Adalimumab & | 0.007 | 7.5 [1.7; 32.7] | 0.018 | 7.4 [1.4; 38.8] | 0.102 | 4.5 [0.7; 27.4] |
| Infliximab | | | | | | |

While HLA-DQA1*05:01G maintained a significant association to responder status in Etanercept and all treatment groups when adjusting for these confounding factors, the association seen with the A allele of rs1600629 was marginally significant in all treatment groups when adjusting for confounding factors; it is not significant for Etanercept or combined Adalimumab and Remicade treatment groups.

### Quantitative Trait Analysis (QTA):

Quantitative trait analyses of the change from baseline in DAS28 score were performed using analysis of covariance. First, the effects of the shared epitope and the presence of allele A at SNP marker rs1800629 in the promoter region of the TNF gene (−308), were assessed as potential confounding factors.

There was no significant association at a type-I error α=0.05 between the number of allele copies of the shared epitope and the change from baseline in DAS28 score in etanercept and in the combined adalimumab and remicade and in the combined treatment groups. There was a significant association with the presence of allele A at SNP marker rs1800629 in the combined adalimumab and remicade (P=0.004) and in the combined treatment groups (P=0.002); the association was however not significant in the subgroup of patients who received etanercept (P=0.214). The presence of allele A at rs1800629 was hence included as covariate in the quantitative trait analyses.

Unadjusted and adjusted analyses on baseline DAS28 score only and on the presence of allele A at rs1800629 were performed to investigate genetic associations between HLA-DQA1*05:01G and the change from baseline in DAS28 score (see Table 6), and similar analysis performed on baseline DAS28 score only and on the presence of allele DQA1*05:01G were performed to investigate genetic associations between the presence of allele A at rs1800629 and the change from baseline in DAS28 score (see Table 7).

TABLE 6
Association results of the presence DQA1*05:01G and the
change from baseline in DAS28 score

| | | | Model adjusted on | | Model adjusted on baseline | |
|---|---|---|---|---|---|---|
| | Univariate model | | baseline DAS28 score | | DAS28 score and TNF rs1800629 | |
| | P | LSMeans (se) | P | LSMeans (se) | P | LSMeans (se) |
| All | 0.098 | Pres.: −1.18 (0.28) | 0.057 | Pres.: −1.20 (0.23) | 0.641 | Pres.: −1.35 (0.23) |
| treatments | | Abs.: −1.76 (0.19) | | Abs.: −1.75 (0.16) | | Abs.: −1.50 (0.18) |
| Etanercept | 0.013 | Pres.: −0.66 (0.42) | 0.021 | Pres.: −0.84 (0.37) | 0.046 | Pres.: −0.83 (0.39) |
| | | Abs.: −2.00 (0.26) | | Abs.: −1.93 (0.23) | | Abs.: −1.94 (0.28) |
| Adalimumab & | 0.780 | Pres.: −1.47 (0.37) | 0.367 | Pres.: −1.32 (0.30) | 0.469 | Pres.: −1.57 (0.29) |
| Remicade | | Abs.: −1.60 (0.26) | | Abs.: −1.66 (0.21) | | Abs.: −1.28 (0.23) |

TABLE 7
Association results of the presence of allele A at rs1800629 and the
change from baseline in DAS28 score

| | | | | | Model adjusted on baseline | |
|---|---|---|---|---|---|---|
| | | | Model adjusted on | | DAS28 score and the presence | |
| | Univariate model | | baseline DAS28 score | | of DQA1*05:01G | |
| | P | LSMeans (se) | P | LSMeans (se) | P | LSMeans (se) |
| All | 0.002 | Pres: −0.90 (0.25) | 0.002 | Pres: −1.02 (0.21) | 0.016 | Pres: −1.04 (0.22) |
| treatments | | Abs: −1.93 (0.18) | | Abs: −1.87 (0.15) | | Abs: −1.81 (0.19) |
| Etanercept | 0.214 | Pres: −0.76 (0.32) | 0.283 | Pres: −1.28 (0.38) | 0.935 | Pres: −1.41 (0.35) |
| | | Abs: −1.98 (0.23) | | Abs: −1.78 (0.26) | | Abs: −1.37 (0.31) |
| Adalimumab & | 0.004 | Pres: −0.75 (0.32) | 0.003 | Pres: −0.88 (0.26) | 0.004 | Pres: −0.84 (0.27) |
| Remicade | | Abs: −1.98 (0.23) | | Abs: −1.91 (0.19) | | Abs: −2.02 (0.24) |

In RES11121, there was perfect correlation between RS9272535 genotype and the presence of HLA-DQA1*05:01G in that all individuals that were homozygous for HLA-DQA1*05:01G were A/A for rs9272535, all individuals that had one copy of HLA-DQA1*05:01G were A/G for rs9272535 and all individuals that did not carry a copy of HLA-DQA1*05:01G were G/G for rs9272535. Review of published HapMap genotype data for rs9272535 and high resolution HLA-DQA1 data (The International HapMap Consortium. The International HapMap Project. Nature 426, 789-796 (2003)) additionally showed 100% correlation between the A allele of rs9272535 and the presence of HLA-DQA1*04, *05 or *06, demonstrating the utility of this marker to identify individuals with these HLA-DQA1 genotypes.

### CONCLUSIONS

This analysis published in this patent shows that specific DNA polymorphisms including rs9272535 may be associated with responsiveness to anti-TNF therapy. The presence of HLA-DQA1*05:01G is associated with non-response to anti-TNF therapies in RES11121, and the observed genetic association is independent of the effect of DAS28 baseline score and TNF −308 genotype (rs1800629) in patients given etanercept. Additionally, the A allele of rs9272535 identifies individuals that are HLA-DQA1*04/*05 and *06 genotype positive (r²=1.00 across RES11121 and HapMap subjects), and can serve as a proxy marker for HLA-DQA1*05:01G genotype. These markers have the potential to identify individuals significantly less likely to respond anti-TNF therapies, particularly etanercept.

A second study was undertaken to replicate this observation in an independent cohort of RA patients from the UK using the Sequenom iPlex platform (www.sequenom.com). This population of RA patients was derived from a UK post marketing register of patients starting anti-TNF therapy run as an industry funded consortium and administered by a UK university. The observational register was established primarily to monitor for safety events, but disease activity assessments were included at registration and at 6 months. The clinical (and subjective) disease activity scores were not mandated to be assessed by independent or consistent observers. Funding decisions on the eligibility of patients to receive funding to initiate and maintain therapy in the UK are based on the disease activity measure, and such biases to disease activity scoring render the data to be less robust than that generated in a clinical trial setting. Relevant disease activity data of the RA patients in the register at baseline and after 6 months of treatment with an anti-TNF inhibitor was used to determine whether the patients responded to their treatment.

DNA from 1,142 patients, recruited by the Biologics in Rheumatoid Arthritis Genetics and Genomics Study Syndicate (BRAGGSS), was used to investigate whether rs9272535 or any of its proxy markers showed a genetic association with response or non-response to anti-TNF therapy (etanercept, infliximab or adalimumab).

A regression approach, with reduction in DAS28 score as the dependent variable, while allowing for baseline DAS28, baselineHAQ score, concurrent DMARD therapy and gender was used to test for genetic effect. We analysed all patients together adjusting for above covariates and treatment, and then each treatment specific subgroup (Inflix, Humira and Enbrel). Patients were then classified as responders (reduction in DAS28 of 1.2 or more at 6 months follow-up) and then using the EULAR classification criteria. Because the primary study, RES11121, had less active RA patients than in the BRAGGS collection, we further evaluated a subset of 266 less severe RA patients for association to improve the similarity of the primary and confirmatory samples.

Suggestive evidence for genetic association with treatment response was observed in the all treatment group, using QT analysis (p=0.037), but no association was found using the responder/non-responder classification (p=0.12). In study RES11121, stronger association was identified in Enbrel treated subjects but this finding is not replicated in these data. Subset analysis of current data indicates that the suggestive signal observed in the all treatments group may be driven mainly by patients on Infliximab (QT analysis, p=0.013, R/NR analysis p=0.011). No association was observed in either the Humira or Enbrel treatment specific subjects. A subset of patients with moderate disease (N=266, baseline DAS28 <=6) in an attempt to create a replicate set that matches that of study RES11121, but could not replicate the previous RES11121 finding.

Our interpretation of this lack of replication in a larger cohort of RA patients when comparing data obtained in a small but highly controlled clinical trial, is that inherent variation and biases in the evaluation of disease activity may have contributed to the loss of the association, and that this study does not invalidate the prior observations.

What is claimed is:

1. The use of HLA-DQA1 as a biomarker for predicting or determining the therapeutic efficacy of anti-TNF therapy.

2. A method for identifying patients that will likely respond or likely not respond to anti-TNF therapy comprising determining the genotype of HLA-DQA1 gene or protein in a patient.

3. A method according to claim 2 wherein the patients have chronic inflammatory disease.

4. A method according to claim 3 wherein the patients have RA.

5. A method of treating inflammation and/or autoimmune disorders in a patient comprising determining the genotype of HLA-DQA1 in a patient followed by administering to the patient an appropriate therapy.

6. A method of treating a patient who is likely to respond to anti-TNF therapy comprising determining the genotype of HLA-DQA1 of a patient followed by administering an anti-TNF therapeutic to said patient.

7. A method of claim 6 wherein the anti-TNF therapeutic is etanercept, infliximab, certolizumad, golimumab and adalimumab.

8. A method for treating a patient who is likely not to respond to anti-TNF therapy comprising determining the genotype of HLA-DQA1 in a patient followed by the administration of a non anti-TNF therapy.

9. A method according to claim 6 wherein the patient has a chronic inflammatory disease.

10. A Method according to claim 9 wherein the patient has RA.

11. A method according to claim 1 wherein the genotype is HLA-DQA1*05:01G genotype and/or carry the A allele for RS9272535.

12. An ex-vivo or in vitro method for determining the HLA-DQ1 genotype of a patient from a sample.

13. A diagnostic kit comprising means for determining the HLA-DQ1 genotype of a patient.
